SEQUENCE LISTING (7 sequences)

SEQ ID NO: 1 - protein, 981 aa, Homo sapiens
MAEGGAADLDTQRSDIATLLKTSLRKGDTWYLVDSRWFKQWKKYVGFDSWDKYQMGDQNVYPGPIDNSGLLKDGDAQSLKEHLIDELDYILLPTEGWNKLVSWYTLMEGQEPIARKVVEQGMFVKHCKVEVYLTELKLCENGNMNNVVTRRFSKADTIDTIEKEIRKIFSIPDEKETRLWNKYMSNTFEPLNKPDSTIQDAGLYQGQVLVIEQKNEDGTWPRGPSTPKSPGASNFSTLPKISPSSLSNNYNNMNNRNVKNSNYCLPSYTAYKNYDYSEPGRNNEQPGLCGLSNLGNTCFMNSAIQCLSNTPPLTEYFLNDKYQEELNFDNPLGMRGEIAKSYAELIKQMWSGKFSYVTPRAFKTQVGRFAPQFSGYQQQDCQELLAFLLDGLHEDLNRIRKKPYIQLKDADGRPDKVVAEEAWENHLKRNDSIIVDIFHGLFKSTLVCPECAKISVTFDPFCYLTLPLPMKKERTLEVYLVRMDPLTKPMQYKVVVPKIGNILDLCTALSALSGIPADKMIVTDIYNHRFHRIFAMDENLSSIMERDDIYVFEININRTEDTEHVIIPVCLREKFRHSSYTHHTGSSLFGQPFLMAVPRNNTEDKLYNLLLLRMCRYVKISTETEETEGSLHCCKDQNINGNGPNGIHEEGSPSEMETDEPDDESSQDQELPSENENSQSEDSVGGDNDSENGLCTEDTCKGQLTGHKKRLFTFQFNNLGNTDINYIKDDTRHIRFDDRQLRLDERSFLALDWDPDLKKRYFDENAAEDFEKHESVEYKPPKKPFVKLKDCIELFTTKEKLGAEDPWYCPNCKEHQQATKKLDLWSLPPVLVVHLKRFSYSRYMRDKLDTLVDFPINDLDMSEFLINPNAGPCRYNLIAVSNHYGGMGGGHYTAFAKNKDDGKWYYFDDSSVSTASEDQIVSKAAYVLFYQRQDTFSGTGFFPLDRETKGASAATGIPLESDEDSNDNDNDIENENCMHTN

SEQ ID NO: 2 - RNA, 17 nt, Artificial Sequence
  siUSP15-1 sense
cuauggaaaugaugaag

SEQ ID NO: 3 - RNA, 17 nt, Artificial Sequence
  siUSP15-1 anti-sense
cuucaucauuuccauag

SEQ ID NO: 4 - RNA, 19 nt, Artificial Sequence
  siUSP15-2 sense
aggaaugagaggugaaaua

SEQ ID NO: 5 - RNA, 19 nt, Artificial Sequence
  siUSP15-2 anti-sense
uauuucaccucucauuccu

SEQ ID NO: 6 - RNA, 19 nt, Artificial Sequence
  siUSP15-3 sense
gcagauaagaugauaguua

SEQ ID NO: 7 - RNA, 19 nt, Artificial Sequence
  siUSP15-3 anti-sense
uaacuaucaucuuaucugc

(19) United States
(12) Patent Application Publication
(10) Pub. No.: US 2015/0344887 A1
(43) Pub. Date: Dec. 3, 2015

(54) siRNA FOR INHIBITION OF USP15 EXPRESSION AND PHARMACEUTICAL COMPOSITION CONTAINING THE SAME

(71) Applicant: KOREA INSTITUTE OF SCIENCE AND TECHNOLOGY, Seoul (KR)

(72) Inventors: Eun Joo SONG, Seoul (KR); Eunice EunKyeong Kim, Seoul (KR); Tanuza Das, Seoul (KR)

(73) Assignee: KOREA INSTITUTE OF SCIENCE AND TECHNOLOGY, Seoul (KR)

(21) Appl. No.: 14/300,590

(22) Filed: Jun. 10, 2014

(30) Foreign Application Priority Data
May 29, 2014 (KR) 10-2014-0065396

(51) Int. Cl. C12N 15/113

(52) U.S. Cl. CPC C12N 15/1137; C12N 2310/14

(57) ABSTRACT

Disclosed herein is an siRNA that inhibits the expression of USP 15 protein and a pharmaceutical composition containing the same for preventing or treating cancers. According to the present invention, the siRNA of the present invention inhibits the expression of USP 15 protein thereby substantially inhibiting the growth and metastasis of cancer cells.

### CROSS-REFERENCE TO RELATED APPLICATION

This application claims under 35 U.S.C. §119(a) the benefit of Korean Patent Application No. 10-2014-0065396 filed May 29, 2014, the entire contents of which are incorporated herein by reference.

### BACKGROUND OF THE INVENTION

1. Field of the Invention

The present invention relates, in general, to SiRNA inhibiting the expression of USP15, and a pharmaceutical composition containing the same.

2. Description of the Related Art

Cell regulation is processed through sophisticated processes such as cell division, growth, differentiation, and apoptosis, and the abnormality in any of the sequential processes causes proliferation of abnormal cells thereby leading to carcinogenesis. Among them, abnormality in cell division is considered the direct cause of carcinogenesis, and thus the substances involved in regulating the process of cell division have been targeted as potent therapeutic agents for preventing and treating cancers.

Each cell division occurs by repeatedly going through cell cycle of G1, S, G2, and M phases, and the cell cycle is regulated by checkpoints present in each cell cycle. The activities of the proteins involved in the cell cycle regulation are mainly controlled by phosphorylation due to kinase and the mechanism of ubiquitine.

Along with ubiquitination, the mechanism of deubiquitination also plays an important role in cell division. For example, Usp44 is involved in the regulation of the anaphase initiation after deubiquitination of cdc20 (Stegmeier F, Rape M, Draviam V M, et al. Anaphase initiation is regulated by antagonistic ubiquitination and deubiquitination activities. Nature 2007; 446:87681), and Usp1 and Usp7 are involved in the regulation of DNA damage checkpoints (Nijman S M, Huang T T, Dirac A M, et al. The deubiquitinating enzyme USP1 regulates the Fanconi anemia pathway. Mol Cell 2005; 17:3319, Huang T T, Nijman S M, Mirchandani K D, et al. Regulation of monoubiquitinated PCNA by DUB autocleavage. Nat Cell Biol 2006; 8:33947). In addition, Usp7 is known to regulate the stability of p53 by deubiquitination of Mdm2 (Brooks C L, Li M, Hu M, Shi Y, Gu W. The p53-Mdm2-HAUSP complex is involved in p53 stabilization by HAUSP. Oncogene 2007; 26:72626), and Usp16 regulates chromosomal segregation during mitosis by deubiquitination of histone H2A (Joo H Y, Zhai L, Yang C, et al. Regulation of cell cycle progression and gene expression by H2A deubiquitination. Nature 2007; 449:106872).

Ubiquitin specific protease 15 (USP15) belongs to a USF family, one of the deubiquitinating enzymes, and includes Cys and His boxes possessed by USP family proteins. Reportedly, USP 15 is involved in the regulation of various cellular functions, and in particular, it is known to suppress the activity of NF-kB by inhibiting TNF-α mediated decomposition via deubiquitinating IkBa (Schweitzer, K., Bozko, P. M., Dubiel, W., and Naumann, M. (2007). CSN controls NF-kappaB by deubiquitinylation of IkappaBalpha. EMBO J. 26, 15321541). Besides, USP15 has been reported to accelerate carcinogenesis by stabilizing TGF-β receptor I via deubiquitination of receptor-activated SMADS (R-SMADS) (Eichhorn, P. J., Rodo′ n, L., Gonzalez-Junca, A., Dirac, A., Gili, M., Martinez-Saez, E., Aura, C., Barba, I., Peg, V., Prat, A., et al. (2012). USP15 stabilizes TGF-beta receptor I and promotes oncogenesis through the activation of TGF-beta signaling in glioblastoma. Nat. Med. 18, 429435. Inui, M., Manfrin, A., Mamidi, A., Martello, G., Morsut, L., Soligo, S., Enzo, E., Moro, S., Polo, S., Dupont, S., et al. (2011). USP15 is a deubiquitylating enzyme for receptor-activated SMADs. Nat. Cell Biol. 13, 13681375). The functional roles of USP15 have been studied as described above, however, no report has elucidated a direct relationship between USP15 and lung cancer.

In this regard, the inventors of the present invention discovered that the expression of USP15 protein is considerably higher in a lung cancer cell line than in normal lung cells, and confirmed that the growth of lung cancer cells was inhibited by suppressing the USP15 expression in a lung cancer cell line using an siRNA, thereby completing the present invention.

Numerous publications and patent documents have been referenced herein with citations as necessary therein over the entire specification. The contents disclosed in the cited references and the patent documents will help clarify the field that the present invention belongs to at a practical level and the constitutional features of the present invention.

### SUMMARY OF THE INVENTION

Accordingly, the inventors of the present invention, while studying cell division regulating materials associated with the proliferation of abnormal cells, discovered that the overexpression of USP15 protein, a USP protein, is directly linked to the growth of cancer cells, and also the blocking of USP15 protein expression by an siRNA can substantially inhibit cancer cell growth, thereby completing the present invention.

Accordingly, an object of the present invention is to provide an siRNA that inhibits the expression of USP15 protein.

Another object of the present invention is to provide a pharmaceutical composition containing the siRNA as an active ingredient for preventing or treating cancers.

The above and other features of the present invention will now be described in detail with reference to certain exemplary embodiments thereof illustrated by the scope of claims, accompanying drawings, which are given hereinbelow by way of illustration only, and thus are not to be understood as limiting the present invention.

In an embodiment of the present invention, there is provided an siRNA that inhibits the expression of USP15 protein. USP15 is a protein that belongs to the Ubiquitin specific protease (USP) family. Being evenly distributed in all tissues, USP15 is involved in the regulation of cellular functions by inducing deubiquitination of various substrates within cells. The inventors of the present invention have discovered that the USP15 protein is overly expressed in cancer cells compared to normal cells, and also that the inhibition of USP15 protein can substantially block the growth of cancer cells.

The USP15 protein of the present invention may include its modified types without change in their activities, preferably the amino acid sequence of SEQ ID NO: 1.

The term, “inhibition of expression” used herein, refers to the removal or reduction in the level of mRNA or protein or both generated from a target gene, which is caused by RNA interference (RNAi) occurring due to mRNA cleavage.

siRNAs may be manufactured by directly synthesizing an siRNA in a test tube and then introducing it into a cell via transfection, or by introducing an siRNA expression vector which is manufactured to express siRNAs in a cell or a PCR-derived siRNA expression cassette into a cell via transfection or infection, or the like. The method of synthesizing an siRNA and introducing it into a cell or an animal may vary depending on the purpose of an experiment or the cellular/biological functions of a gene product.

The term, “siRNA” used herein, refers to a nucleic acid molecule that can mediate RNA interference or gene silencing. The siRNA used in the present invention may include a sequence complementary to the mRNA of USP15 protein. The term, “complementary” used herein, may include, in addition to a case with 100% complementarity, other cases with incomplete complementarity that may inhibit the expression of USP15 gene via the mechanism of RNA interference, preferably 90% complementarity, more preferably 98% complementarity, and most preferably 100% complementarity. Preferably, the entire length of the siRNA for USP15 may be in the range of from 10 to 60 nucleotides (nt), more preferably, from 15 to 30 nt.

In a preferred embodiment of the present invention, the siRNA of the present invention may have a nucleotide sequence complementary to a partial mRNA sequence for USP15 having a SEQ ID NO: 1. The partial mRNA sequence may be a sequence valid for mediating silencing of USP15 gene with a size of at least 10 nt, preferably at least 15 nt, more preferably at least 15 nt.

The siRNA molecule of the present invention may include a nucleotide sequence with the shortest length described above or a sequence complementary to the sequence, preferably at most 60 nt, more preferably at most 30 nt.

In a preferred embodiment of the present invention, the siRNA molecule of the present invention may include a sense sequence of SEQ ID NO: 2, SEQ ID NO: 4, or SEQ ID NO: 6; and an anti-sense sequence of SEQ ID NO: 3, SEQ ID NO: 54, or SEQ ID NO: 7.

Both a blunt end and a cohesive end may be acceptable as an siRNA terminal structure as long as they can inhibit the expression of a target gene via RNAi effect. The terminal structure with a cohesive end may have a protruding 3′ terminus or a protruding 5′ terminus, and there is no limit on the number of protruding nucleotides. For example, the number of nucleotides may be in the range of from 1 to 8 nt, preferably, from 2 to 6 nt. Additionally, the siRNA may include, within the range capable of maintaining the inhibitory effect of a target gene expression, a low molecular weight RNA (e.g., a natural RNA molecule such as tRNA, rRNA, and viral RNA or an artificial RNA molecule) on a protruding portion of one terminus. The siRNA terminal structure may not necessarily be truncated at both termini but it may be a stem loop structure in which a terminal portion of one of a double stranded RNA is connected via a linker RNA. There is no special limit on length of the linker as long as it is sufficient to form a pair in the stem loop portion.

The siRNA of the present invention may include at least one modified form of a functional equivalent such as a substitution, an insertion, a deletion, or a combination thereof, in which the change does not decrease the activity of the siRNA. The modified form may have a sequence homology of 70% or higher to those of the siRNA (SEQ ID NO: 2 to SEQ ID NO: 7), preferably 80%, more preferably 90%, and most preferably 95% or higher. The homology may be easily determined by comparing the sequence of a polynucleotide with a corresponding portion of a target polynucleotide using an algorithm widely known in the related art, for example, Align or BLAST algorithm (Altschul, S. F. J. Mol. Biol. 219, 555-565, 1991; Henikoff, S. and Henikoff, J. G. Proc. Natl. Acad. Sci. USA 89, 10915-10919, 1992).

The siRNA of the present invention may be used in any cell where USP15 protein is expressed in order to suppress the expression of USP15 protein, preferably, to prevent the growth of cancer cells.

In an another aspect of the present invention, there is provided a pharmaceutical composition containing an siRNA, which inhibits the expression of USP15 within a cell via a complementary binding to the nucleotide sequence of a transcriptome of USP15 protein, as an active ingredient.

The siRNA of the present invention may be administered as a pharmaceutical composition in general for preventing or treating of cancers. The administration may be performed by a known method by which a nucleic acid is introduced into a target cell in vivo or in vitro. The method of introducing the siRNA into a cell may not be particularly limited. Preferably, the siRNA may be directly sunk into a host cell, or introduced after transfecting the host cell using a recombinant vector. The expression vector to be used is not particularly limited.

The recombinant vector that expresses the siRNA may include a plasmid or a viral vector selected from the group consisting of an adeno-associated virus, a retrovirus, a vaccinia virus, and an oncolytic virus. Meanwhile, a conventional gene transfer technology may include transfection using calcium phosphate, DEAE-dextran, electroporation, and microinjection, and viral vector (Graham, F. L. and van der Eb, A. J. (1973) Virol. 52, 456; McCutchan, J. H. and Pagano, J. S. (1968), J. Natl. Cancer Inst. 41, 35 1; Chu, G. et al. (1987), Nucl. Acids Res. 15, 1311; Fraley, R. et al. (1980), J. Biol. Chem. 255, 10431; Capecchi, M. R. (1980), Cell 22, 479). Recently, a method using a cationic liposome has been added thereto to introduce nucleotide into a cell (Felgner, P. L. et al. (1987), Proc. Nati. Acad. Sci. USA 84, 7413). Examples of commercially available cationic lipid preparations include Tfx 50(Promega) and Lipofectamine 2000 (Life Technologies). A method of directly sinking an siRNQA which can complementarily bind to the USP15 gene may not be particularly limited, but preferably, the sinking may be performed by mixing 2 to 6 μg of cationic liposome with 1 μg of siRNA followed by lipofection for from 15 to 40 minutes.

The pharmaceutical composition of the present invention may further include a pharmaceutically acceptable carrier or excipient. Pharmaceutically acceptable excipients are widely known in the related art, and are relatively inert materials that help in the easy administration of a pharmaceutically active material. For example, an excipient may provide a shape or a viscosity, and may serve as a diluent. Examples of a suitable excipient may include a stabilizing agent, a humectant, an emulsifier, salts that can change osmolarity, an encapsulant, a buffer solution, a skin penetration promoter, but are not limited thereto. Excipients and formulations for oral and parenteral drug delivery have been suggested (Remington, The Science and Practice of Pharmacy 20^{th} edition, Mack Publishing (2000).

In an another embodiment of the present invention, there is provided a method for treating cancers including administering an effective amount of an siRNA, which can inhibit the USP15 expression in a cell via a complementary binding to the nucleotide sequence of transcriptome of the USP15 protein.

An effective amount of the siRNA is the amount for obtaining the intended result, for example, the amount sufficient for providing a reduced level of the expression of USP15 gene compared to the normal expression detected in the absence of the siRNA. The siRNA may be introduced in an amount which may allow the delivery of at least a single copy per each cell. The greater the amount of copies (for example, 5 or more copies, 10 or more copies, 100 or more copies, or 1,000 or more copies per each cell, respectively), the higher the inhibition efficiency, and one with a lesser amount of introduction may be more advantageous in a particular application.

The term, “administration” used herein, refers to the introduction of a particular material to a patient in any appropriate way, and the administration of a substance may be performed via a general route as long as it can be arrived at the target tissue. The administration may be performed via an intraperitoneal administration, intravenous administration, intramuscular administration, intradermal administration, oral administration, topical administration, intranasal administration, intrapulmonoary administration, and rectal administration, but are not limited thereto. Additionally, the pharmaceutical composition may be administered by any apparatus in which an active material can move to a target cell.

Furthermore, the term “administration” used herein, refers to the introduction of the siRNA of the present invention to a cell which expresses the USP15 gene via “systemic delivery” or “topical delivery”. “Systemic delivery” refers to a delivery that induces a wide range of biodistribution in an organism. Some technologies for administration may induce systemic delivery on a particular substance but may not induce other substances. Systemic delivery means that a useful, preferably a therapeutically effective amount of an siRNA, is exposed on most parts of a given body. Generally, to obtain a wide range of biodistribution, it is essential that the siRNA be sustained in blood until it reaches the target area distantly located from the administration site without being instantly decomposed or removed (for example, by passing through body organs such as the liver and lungs, or by a prompt non-specific cell binding). The systemic delivery of nucleic acid lipid particles may be performed by any known method in the art (e.g., intravenous, subcutaneous, intra-abdominal deliveries, etc.). In a preferred embodiment of the present invention, the systemic delivery of a nucleic acid particle may be performed via intravenous delivery. The term, “topical delivery” used herein, refers to a direct delivery of the siRNA to the target area in an organism. For example, the siRNA may be topically delivered to the area of a disease (e.g., a tumorigenic area or other targeted areas (e.g., an area of inflammation or a target organ (e.g., liver, heart, pancreas, kidney, etc.)) by a direct injection.

### Advantageous Effects

The advantages of the present invention may be summarized as follows.

(i) The present invention provides an siRNA that can inhibit the expression of USP15 protein.

(ii) The present invention also provides a pharmaceutical composition containing the siRNA as an active ingredient for preventing or treating cancers.

(iii) The siRNA of the present invention can block the over-expression of the USP15 protein, thereby considerably inhibiting the growth of cancer cells.

(iv) The siRNA of the present invention can block the over-expression of the USP15 protein, thereby considerably inhibiting the metastasis of cancer cells.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a result of western blot analysis showing the overexpression of USP15 protein in non-small cell lung cancer cells (NCI-H157 and NCI-H358), as compared with the USP15 protein expression in a normal lung cell (WI-38).

FIG. 2 is a result of western blot analysis showing the inhibition of USP15 protein expression by siRNA.

FIG. 3 is a graph showing the inhibition of cancer cell growth in NCI-H157, a non-small cell lung cancer cell, according to the decrease in the expression of USP15 protein.

FIG. 4 shows pictures of cell migration inhibition in NCI-H157, a non-small cell lung cancer cell, according to the decrease in USP15 protein expression via scratch assay.

FIG. 5A is a picture showing the inhibition of cell invasion in NCI-H157, a non-small cell lung cancer cell, according to the decrease in USP15 protein expression.

FIG. 5B is a graph showing a scaled inhibition of cell invasion in NCI-H157, a non-small cell lung cancer cell, according to the decrease in USP15 protein expression.

### DESCRIPTION OF THE PREFERRED EMBODIMENTS

A better understanding of the present invention may be obtained through the following examples which are set forth to illustrate, but are not to be construed as the limit of the present invention.

### EXAMPLES

### Example 1

### Confirmation of Over-Expression of USP15 Protein in a Lung Cancer Cell Line

The expression pattern of USP15 protein was examined using WI-38, a normal lung cell, and NCI-H358, a non-small cell lung cancer cell.

The WI-38 cells (normal lung cells), and NCI-H358 and NCI-H157 cells (non-small cell lung cancer cells), were cultured in an RPMI medium containing 10% FBS (Gibco/BRL), 100 units/mL of penicillin, 100 μg/mL streptomycin (Gibco/BRL) at 37° C. and 5% CO₂.

The cultured cells were aliquoted into a 6-well plate in the amount of 2×10⁵ cells/well and cultured. Twenty four hours later, the cultured cells were washed 3 times with a cold PBS, and lysed using a cell lysate containing a lysis buffer, 50 mM Tris-Cl, pH 7.4, 150 mM NaCl, 2 mM EDTA, 0.5% NP-40, protease inhibitor cocktail, and 1 mM sodium orthovanadate. After centrifugation, the supernatant was recovered and the amount of the total proteins was measured using a micro BCA protein analysis kit.

An equal amount of a protein was separated via an SDS-PAGE gel electrophoresis and then electrically transferred onto a nitrocellulose membrane. The membrane was reacted with antibodies of USP15 protein (Bethyl Laboratories, Inc.).

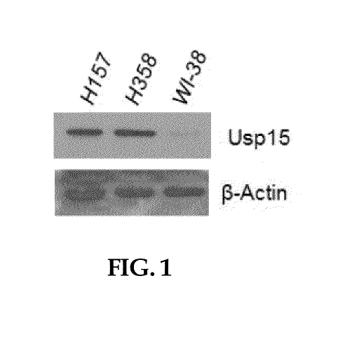

From the results shown in FIG. 1 comparing the USP15 expression in WI-38 cells (normal lung cells) with those in NCI-H157 and NCI-H358 cells (non-small cell lung cancer cells), it was confirmed that the USP15 expression was much higher in NCI-H157 and NCI-H358 cells than in WI-38 cells.

### Example 2

### Inhibitory Effect of USP15 siRNA on Protein Expression

Upon preparation an siRNA for USP15, it was introduced into NCI-H157 cells, a non-small cell lung cancer cell line, and examined the siRNA's inhibitory effect against protein expression.

The NCI-H157 cells, as a non-small cell lung cancer cell line, were aliquoted into a 6-well plate in the amount of 2×10⁵ cells/well and cultured for one day. The cultured cells were transfected using three different kinds of siRNAs shown in Table 1, a control siRNA (NC siRNA) (Bioneer Co., Korea), and 50 nM of lipofectamine reagent (Invitrogen). Two days after the transfection, the inhibitory effect of the siRNAs against USP15 expression was confirmed via western blotting.

TABLE 1

| | Sense | Anti-sense |
|---|---|---|
| siUSP15-1 | 5′-CUAUGGAAAU | 5′-CUUCAUCAUU |
| | GAUGAAG-3′ | UCCAUAG-3′ |
| | (SEQ ID NO: 2) | (SEQ ID NO: 3) |
| siUSP15-2 | 5′-AGGAAUGAGA | 5′-UAUUUCACCU |
| | GGUGAAAUA-3′ | CUCAUUCCU-3′ |
| | (SEQ ID NO: 4) | (SEQ ID NO: 5) |
| siUSP15-3 | 5′-GCAGAUAAGA | 5′-UAACUAUCAU |
| | UGAUAGUUA-3′ | CUUAUCUGC-3′ |
| | (SEQ ID NO: 6) | (SEQ ID NO: 7) |

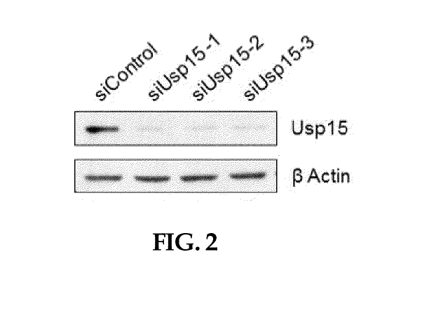

As shown in FIG. 2, it was confirmed that the transfection of siRNAs of USP15 in NCI-H157 cells reduced the expression of USP15 proteins.

### Example 3

### Inhibition of Growth of Non-Small Cell Lung Cancer Cells Via Introduction of USP15 siRNA

The effect of the introduction of USP15 siRNA on cell growth was examined by analyzing cell numbers according to time. The NCI-H157 cells, as a non-small cell lung cancer cell line, were aliquoted into a 12-well plate in the amount of 1×10⁴ cells/well and cultured for one day. The cultured cells were transfected using the same three different kinds of siRNAs means as in Example 2, a control siRNA (NC siRNA) (Bioneer Co., Korea), and 50 nM of lipofectamine reagent (Invitrogen). The effect of USP15 on the growth of the non-small cell lung cancer cell line was examined by counting the number of cells 24, 36, 48, 60, and 72 hours after the transfection by a hemocytometer.

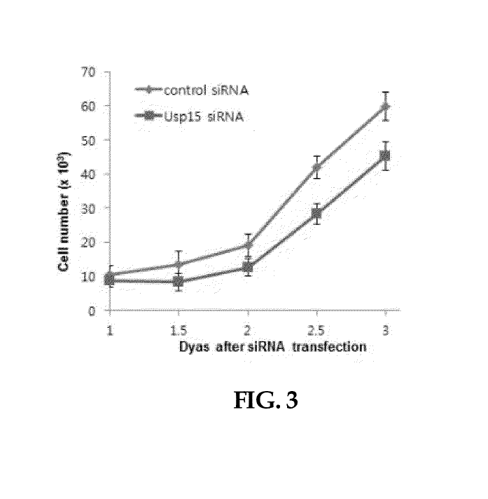

As a result, it was confirmed that the introduction of USP15 siRNAs into NCI-H157 cells reduced the cell growth as shown in FIG. 3.

### Example 4

### Inhibition of Migration of Non-Small Cell Lung Cancer Cells Via Introduction of USP15 siRNA

The effect of the introduction of USP15 siRNA on cell migration was examined via an in vitro scratch assay (Liang CCl, Park A Y, Guan J L. In vitro scratch assay: a convenient and inexpensive method for analysis of cell migration in vitro. Nat Protoc. 2007; 2(2):329-33.).

The NCI-H157 cells, as a non-small cell lung cancer cell line, were aliquoted into a 6-well plate in the amount of 4×10⁵ cells/well and cultured for one day. The cultured cells were transfected using the same three different kinds of siRNAs me as in Example 2, a control siRNA (NC siRNA) (Bioneer Co., Korea), and 50 nM of lipofectamine reagent (Invitrogen). Upon transfection, the cells were cultured until they formed a 100% confluence as a monolayer, and the monolayer was scratched off by generating scratches on the cells in a straight line using a p200 pipette. Then, 24 hours thereafter, the level filled-in with scratch lines were pictured under a microscope and compared.

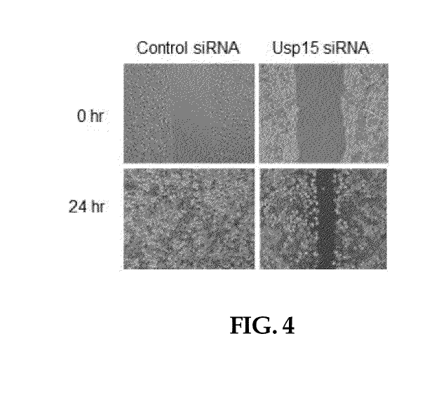

As a result, it was confirmed that the introduction of the USP15 siRNA in NCI-H157 cells reduced the cell migration as shown in FIG. 4.

### Example 5

### Inhibition of Invasion of Non-Small Cell Lung Cancer Cells Via Introduction of USP15 siRNA

The effect of the introduction of USP15 siRNA on cell invasion was examined via matrigel invasion assay.

The NCI-H157 cells, as a non-small cell lung cancer cell line, were aliquoted into a 6-well plate in the amount of 4×10⁵ cells/well and cultured for one day. The cultured cells were transfected using the same three different kinds of siRNAs means as in Example 2, a control siRNA (NC siRNA) (Bioneer Co., Korea), and 50 nM of lipofectamine reagent (Invitrogen), and cultured for 48 hours. While culturing, matrigel was prepared for the subsequent step. Matrigel was dissolved at 4° C., and then diluted in 1 mg/mL of a serum-free media. Then, 100 μL of the diluted matrigel was added into a 24-well transwell and solidified at 37° C. for 4 to 5 hours. The transfected cells were added into a medium containing 1% FBS to a final concentration of 10⁶ cells/mL, and 100 μL of the cell suspension was placed on top of the matrigel. Additionally, a lower chamber of the chamber was filled with a medium containing 600 uL of 5 μg/mL fibronectin, and cultured at 37° C. for 24 hours. After removing the transwell, the cells were dyed with a diff-quick solution. All the uninvaded cells were removed using cotton swabs, and the number of the invaded cells on the lower surface was counted under a microscope.

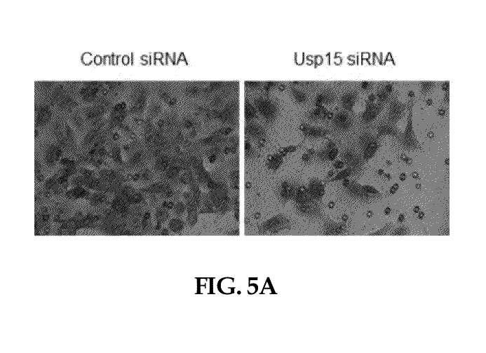

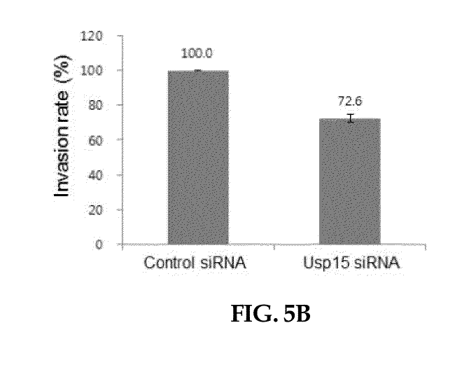

As a result, it was confirmed that the introduction of the USP15 siRNA in NCI-H157 cells reduced the cell invasion as shown in FIGS. 5A and 5B.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

### SEQUENCE LISTING TEXT

(C:\KipoNet\KEditor\appfile\DP-2014-0268_KIST_siRNA.app)

What is claimed is:

1. A pharmaceutical composition comprising siRNA, which inhibits the expression of USP15 protein in cells via a complementary binding to the SEQ ID NO: 1 of the transcriptome sequence of the USP15 protein, as an active ingredient for preventing or treating cancers.

2. The pharmaceutical composition of claim 1, wherein the USP15 protein is overexpressed in a cancer cell compared to a normal cell.

3. The pharmaceutical composition of claim 2, wherein the cancer cell is a lung cancer cell.

4. The pharmaceutical composition of claim 1, wherein the siRNA comprises a sense sequence of SEQ ID NO: 2 and an anti-sense of SEQ ID NO: 3.

5. The pharmaceutical composition of claim 1, wherein the siRNA comprises a sense sequence of SEQ ID NO: 4 and an anti-sense of SEQ ID NO: 5.

6. The pharmaceutical composition of claim 1, wherein the siRNA comprises a sense sequence of SEQ ID NO: 6 and an anti-sense of SEQ ID NO: 7.

7. An siRNA comprising a sense sequence of SEQ ID NO: 2, SEQ ID NO: 4, or SEQ ID NO: 6 or an anti-sense sequence of SEQ ID NO: 3, SEQ ID NO: 5, or SEQ ID NO: 7, which inhibits the expression of USP15 protein.

8. The siRNA of claim 7, wherein the USP15 protein is overexpressed in a cancer cell compared to a normal cell.

9. The siRNA of claim 8, wherein the cancer cell is a lung cancer cell.

10. A pharmaceutical composition comprising the siRNA of claim 7 for preventing or treating cancers.
